(19) United States
(12) Patent Application Publication
(10) Pub. No.: US 2026/0154820 A1
(43) Pub. Date: Jun. 4, 2026

(54) MEDICAL IMAGE DIAGNOSTIC APPARATUS

(71) Applicant: CANON MEDICAL SYSTEMS CORPORATION, Otawara-shi (JP)

(72) Inventor: Shuki MARUYAMA, Nasushiobara Tochigi (JP)

(73) Assignee: CANON MEDICAL SYSTEMS CORPORATION, Otawara-shi (JP)

(21) Appl. No.: 19/406,035

(22) Filed: Dec. 2, 2025

(30) Foreign Application Priority Data
Dec. 3, 2024 (JP) 2024-210657

(51) Int. Cl. G06T 7/00; A61B 5/00; G06T 7/73

(52) U.S. Cl. CPC G06T 7/0016; A61B 5/0037; A61B 5/0077; A61B 5/7267; G06T 7/74; G06T 2207/20081

(57) ABSTRACT

A medical image diagnostic apparatus includes an acquisition unit, a setting unit, an identification unit, an imaging control unit, and an output unit. The acquisition unit acquires an image captured in preparatory imaging before main imaging in an imaging examination of a subject and finding information. The setting unit sets a main imaging region including a plurality of slices to be captured in the main imaging based on the image captured in the preparatory imaging. The identification unit identifies the position of an abnormal slice from among the plurality of slices included in the main imaging region based on the image captured in the preparatory imaging and the finding information. The imaging control unit performs the main imaging to capture an image of the main imaging region. The output unit outputs the image captured in the main imaging together with abnormal slice information about the position of the abnormal slice.

### CROSS-REFERENCE TO RELATED APPLICATIONS

This application is based upon and claims the benefit of priority from Japanese Patent Application No. 2024-210657, filed Dec. 3, 2024, the entire contents of which are incorporated herein by reference.

### FIELD

Embodiments described herein relate generally to a medical image diagnostic apparatus.

### BACKGROUND

Diagnostic processes in medical practice have conventionally been performed in the following order: a medical interview and a physical examination are conducted on a subject, examinations such as an imaging examination are conducted as needed, and then a diagnosis is made.

In the medical interview and the physical examination, a site of pain of the body of the subject is checked because there is a correlation between a site of pain and a disease, and thus a site with a suspicion of disease is estimated. Generally, in the imaging examination, an image is captured so as to include not only an image of the site with a suspicion of disease estimated in the medical interview and the physical examination, but also an image of a peripheral region of the site with a suspicion of disease.

Specifically, in the imaging examination, a medical image diagnostic apparatus is used to perform preparatory imaging, including capturing of a positioning image and the like, and then perform main imaging for capturing an image of a main imaging region, which is set so as to include the site with a suspicion of disease and the peripheral region thereof, based on an image captured in preparatory imaging.

Subsequently, in the imaging examination, the medical image diagnostic apparatus or an interpretation terminal device is used to identify an abnormal slice by searching for a slice including an abnormal site in an image captured in main imaging, and further by checking findings obtained through the medical interview and the physical examination, to determine whether there is a problem with imaging conditions, whether there is a need to perform additional imaging, or whether to terminate the examination.

As described above, in the imaging examination, a wider main imaging region is generally set so that the main imaging region can include the site with a suspicion of disease and the peripheral region thereof, and thus the image captured in main imaging includes a large number of slices. As a result, it takes a long time to identify an abnormal slice.

Accordingly, in medical practice, there is a demand for reducing the time required for identifying an abnormal slice.

### BRIEF DESCRIPTION OF THE DRAWINGS

FIG. 1 is a diagram illustrating a configuration example of a medical image processing system including a medical image diagnostic apparatus according to a first embodiment;

FIG. 2 is a diagram illustrating a configuration example of the medical image diagnostic apparatus according to the first embodiment;

FIGS. 3A to 3C each illustrate an example of processing to be performed by each processing function included in processing circuitry of the medical image diagnostic apparatus according to the first embodiment;

FIGS. 4A and 4B each illustrate a first example of the medical image diagnostic apparatus according to the first embodiment;

FIGS. 5A and 5B each illustrate a second example of the medical image diagnostic apparatus according to the first embodiment;

FIGS. 6A and 6B each illustrate a third example of the medical image diagnostic apparatus according to the first embodiment;

FIGS. 7A to 7E each illustrate a fifth example of the medical image diagnostic apparatus according to the first embodiment;

FIGS. 8A to 8D each illustrate a sixth example of the medical image diagnostic apparatus according to the first embodiment;

FIGS. 9A and 9B each illustrate a seventh example of the medical image diagnostic apparatus according to the first embodiment; and

FIG. 10 is a flowchart illustrating a procedure of processing to be performed by each processing function included in the processing circuitry of the medical image diagnostic apparatus according to the first embodiment.

### DETAILED DESCRIPTION

A medical image diagnostic apparatus according to an embodiment includes an acquisition unit, a setting unit, an identification unit, an imaging control unit, and an output unit. The acquisition unit acquires an image captured in preparatory imaging before main imaging in a same imaging examination of a subject and finding information. The setting unit sets a main imaging region including a plurality of slices to be captured in the main imaging based on the image captured in the preparatory imaging. The identification unit identifies the position of an abnormal slice from among the plurality of slices included in the main imaging region based on the image captured in the preparatory imaging and the finding information. The imaging control unit performs the main imaging to capture an image of the main imaging region. The output unit outputs the image captured in the main imaging together with abnormal slice information about the position of the abnormal slice.

Various Embodiments will be described hereinafter with reference to the accompanying drawings.

### First Embodiment

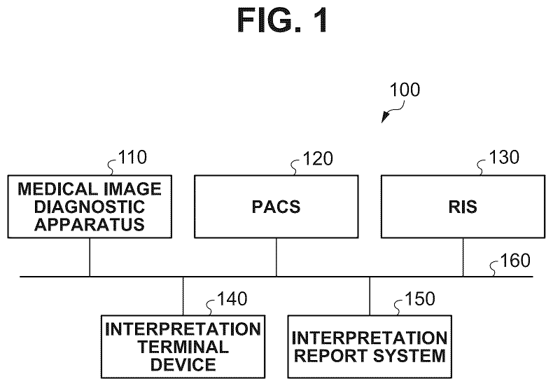

FIG. 1 is a diagram illustrating a configuration example of a medical image processing system including a medical image diagnostic apparatus according to a first embodiment.

For example, as illustrated in FIG. 1, a medical image processing system 100 includes a medical image diagnostic apparatus 110, a picture archiving and communication system (PACS) 120, a radiology information system (RIS) 130, an interpretation terminal device 140, and an interpretation report system 150. Herein, these systems, the apparatus, and the device are communicably interconnected via a network 160 such as a wired network or a wireless network.

The medical image diagnostic apparatus 110 captures images of a subject and collects medical images. The medical image diagnostic apparatus 110 is, for example, a magnetic resonance imaging (MRI) apparatus, an X-ray computed tomography (CT) apparatus, a positron emission tomography (PET)-CT apparatus, or a single photon emission computed tomography (SPECT)-CT apparatus.

The PACS 120 stores various medical images of the subject. Specifically, the PACS 120 acquires various medical images from the medical image diagnostic apparatus 110 via the network 160, and stores the acquired medical images therein. Further, the PACS 120 acquires finding information from the interpretation terminal device 140 and the interpretation report system 150 via the network 160, adds the acquired finding information to the medical images on which the finding information is based as supplementary information, and stores the finding information. For example, the PACS 120 is implemented by a computer device such as a server or a workstation.

The RIS 130 stores various medical information about the subject. Specifically, the RIS 130 acquires medical images obtained through an imaging examination of the subject and finding information from the PACS 120 via the network 160, and stores the acquired medical images and finding information. The RIS 130 receives input of examination results obtained through various examinations, such as a blood test, and finding information from an operator, and stores the input examination results and finding information. For example, the PACS 120 is implemented by a computer device such as a server or a workstation.

The interpretation terminal device 140 creates an interpretation report and finding information about the subject. Specifically, the interpretation terminal device 140 acquires medical images from the medical image diagnostic apparatus 110 and the PACS 120 via the network 160 in response to a request from the operator, and displays the acquired medical images on a display. Further, the interpretation terminal device 140 receives input of interpretation results on the medical images displayed on the display from the operator, and creates an interpretation report based on the input interpretation results. The interpretation terminal device 140 also receives input of findings about the medical images displayed on the display from the operator, and generates finding information based on the input findings. Further, the interpretation terminal device 140 transmits the created interpretation report and finding information to the interpretation report system 150. For example, the interpretation terminal device 140 is implemented by a computer device such as a personal computer or a tablet terminal.

The interpretation report system 150 stores the interpretation report and finding information about the subject. Specifically, the interpretation report system 150 receives the interpretation report and finding information transmitted from the interpretation terminal device 140 via the network 160, and stores the received interpretation report and finding information therein. For example, the interpretation report system 150 is implemented by a computer device such as a server or a workstation.

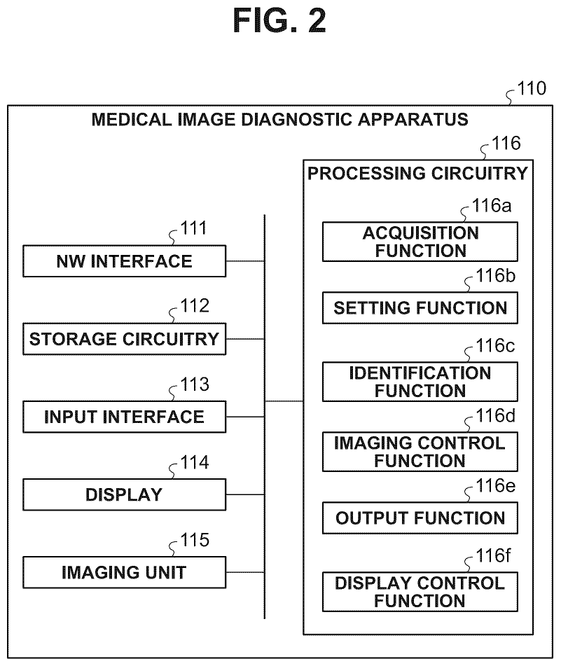

FIG. 2 is a diagram illustrating a configuration example of the medical image diagnostic apparatus 110 according to the first embodiment.

For example, as illustrated in FIG. 1, the medical image diagnostic apparatus 110 includes a network (NW) interface 111, storage circuitry 112, an input interface 113, a display 114, an imaging unit 115, and processing circuitry 116.

The NW interface 111 controls transmission and communication of various kinds of data to be transmitted and received between another device connected via the network 160. Specifically, the NW interface 111 is connected to the processing circuitry 116 and controls transmission and communication of various kinds of data under control of the processing circuitry 116. For example, the NW interface 111 is implemented by a network card, a network adapter, or a network interface controller (NIC).

The storage circuitry 112 stores various kinds of data, various programs, and the like. Specifically, the storage circuitry 112 is connected to the processing circuitry 116 and stores and reads out various kinds of data, various programs, and the like under the control of the processing circuitry 116. For example, the storage circuitry 112 is implemented by a semiconductor memory element such as a random access memory (RAM) or a flash memory, a hard disk, or an optical disk.

The input interface 113 receives input operations of various instructions and various kinds of information from the operator. Specifically, the input interface 113 is connected to the processing circuitry 116, converts the input operation received from the operator into an electric signal, and outputs the electric signal to the processing circuitry 116. For example, the input interface 113 is implemented by a mouse, a keyboard, a touch-pad on which an input operation is performed by touching on an operation surface, a touch screen in which a display screen and a touch-pad are integrated, non-contact input circuity using an optical sensor, or voice input circuitry.

The display 114 displays various kinds of information and various kinds of data. Specifically, the display 114 is connected to the processing circuitry 116 and displays various kinds of information and various kinds of data output from the processing circuitry 116. For example, the display 114 is implemented by a liquid crystal monitor, a cathode ray tube (CRT) monitor, or a touch panel, or the like.

The imaging unit 115 generates various medical images by capturing images of the subject based on imaging conditions input from the operator. Further, the imaging unit 115 causes the storage circuitry 112 to store the generated medical images.

For example, in a case where the medical image diagnostic apparatus 110 is an MRI apparatus, the imaging unit 115 includes a gantry device including a static magnetic field magnet, a gradient magnetic field coil, a gradient magnetic field power supply, a transmission radio frequency (RF) coil, a reception RF coil, transmitter circuitry, and receiver circuitry, a couch device on which a subject is placed, processing circuitry including an image reconstruction function, and the like. The imaging unit 115 collects magnetic resonance data from the subject and generates an MR image.

For example, in a case where the medical image diagnostic apparatus 110 is an X-ray CT apparatus, the imaging unit 115 includes a gantry device including an X-ray tube, an X-ray detector, a rotary frame that causes the X-ray tube and the X-ray detector to be positioned to face each other and to move on a circumferential orbit, and a data acquisition system (DAS), a couch device on which the subject is placed, processing circuitry including the image reconstruction function, and the like. The imaging unit 115 detects X-rays transmitted through the subject and generates a CT image.

For example, in a case where the medical image diagnostic apparatus 110 is a PET-CT apparatus, the imaging unit 115 includes a PET imaging unit configured to generate a PET image by detecting annihilation gamma rays simultaneously emitted in opposite directions from a radiopharmaceutical administered to the subject, and a CT imaging unit configured to generate a CT image by detecting X-rays transmitted through the subject. The imaging unit 115 generates a PET image, a CT image, and an image obtained by combining the PET image and the CT image.

For example, in a case where the medical image diagnostic apparatus 110 is a SPECT-CT apparatus, the imaging unit 115 includes a SPECT imaging unit configured to generate a SPECT image by detecting gamma rays emitted in one direction from a radiopharmaceutical administered to the subject, and a CT imaging unit configured to generate a CT image by detecting X-rays transmitted through the subject. The imaging unit 115 generates a SPECT image, a CT image, and an image obtained by combining the SPECT image and the CT image.

The processing circuitry 116 controls constituent elements of the medical image diagnostic apparatus 110, thereby controlling the entire medical image diagnostic apparatus 110. Specifically, the processing circuitry 116 displays a graphical user interface (GUI) for receiving an input operation of various instructions and various kinds of information from the operator on the display 114, and controls the constituent elements of the medical image diagnostic apparatus 110 according to the input operation received through the input interface 113. For example, the processing circuitry 116 receives input of imaging conditions from the operator and controls the imaging unit 115 based on the input imaging conditions to capture an image of the subject and generate a medical image. Further, the processing circuitry 116 causes the storage circuitry 112 to store the generated medical image, reads out the medical image stored in the storage circuitry 112 in response to a request from the operator, and causes the display 114 to display the medical image.

Configuration examples of the medical image processing system 100 and the medical image diagnostic apparatus 110 according to the first embodiment have been described above. The medical image processing system 100 and the medical image diagnostic apparatus 110 according to the present embodiment are installed in, for example, a medical facility such as a hospital or a medical clinic, and is used for diagnosis of various diseases made by a doctor or the like.

In general, diagnostic processes in medical practice are performed in the following order: a medical interview and a physical examination are conducted on a subject, examinations such as an imaging examination are conducted as needed, and then a diagnosis is made.

In the medical interview and the physical examination, since there is a correlation between a site of pain and a disease, a site with a suspicion of disease is estimated by checking the site of pain of the subject. Generally, in the imaging examination, an image is captured so as to include not only an image of the site with a suspicion of disease estimated in the medical interview and the physical examination but also an image of a peripheral region of the site with a suspicion of disease.

Specifically, in the imaging examination, a medical image diagnostic apparatus is used to perform preparatory imaging, including capturing of a positioning image and the like, and then perform main imaging to capture an image of a main imaging region that is set so as to include the site with a suspicion of disease and the peripheral region thereof, based on the image captured in preparatory imaging.

Subsequently, in the imaging examination, the medical image diagnostic apparatus or an interpretation terminal device is used to identify an abnormal slice by searching for a slice including an abnormal site in the image captured in the main imaging, and further check findings obtained through the medical interview and the physical examination, thereby determining whether there is a problem with imaging conditions, whether there is a need to perform additional imaging, or whether to terminate the examination.

As described above, in general, in the imaging examination, the main imaging region is widely set so that the main imaging region can include the site with a suspicion of disease and the peripheral region thereof, and thus the image captured in main imaging includes a large number of slices. As a result, it takes a long time to identify an abnormal slice.

In view of the above, in the present embodiment, the medical image diagnostic apparatus 110 is configured to reduce the time required for identifying an abnormal slice.

Specifically, the processing circuitry 116 in the medical image diagnostic apparatus 110 includes an acquisition function 116a, a setting function 116b, an identification function 116c, an imaging control function 116d, an output function 116e, and a display control function 116f.

In this case, the acquisition function 116a is an example of an acquisition unit. The setting function 116b is an example of a setting unit. The identification function 116c is an example of an identification unit. The imaging control function 116d is an example of an imaging control unit. The output function 116e is an example of an output unit. The display control function 116f is an example of a display control unit.

Processing functions included in the processing circuitry 116 of the medical image diagnostic apparatus 110 will be described in detail below.

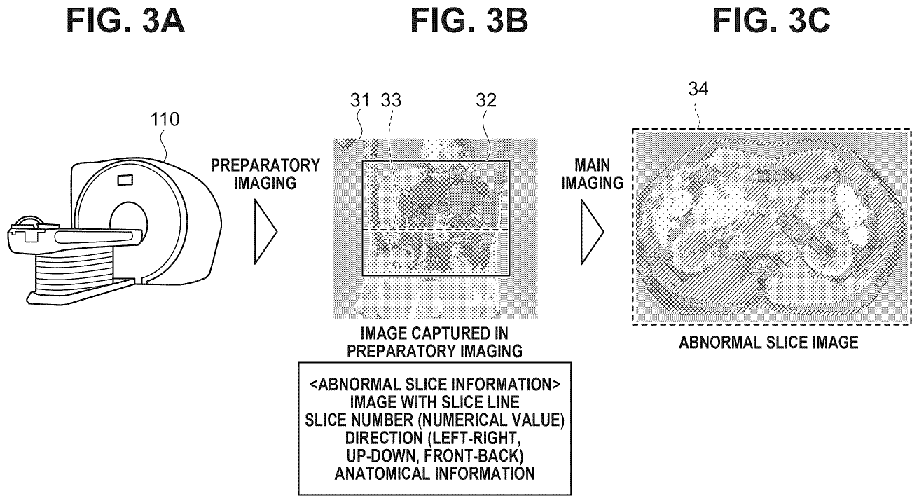

FIGS. 3A to 3C each illustrate an example of processing to be performed by each processing function included in the processing circuitry 116 of the medical image diagnostic apparatus 110 according to the first embodiment.

The acquisition function 116a acquires an image captured in preparatory imaging and finding information obtained before main imaging in an imaging examination of a subject to be diagnosed.

For example, as illustrated in FIG. 3A, the acquisition function 116a acquires an image 31 captured in preparatory imaging of the subject by the medical image diagnostic apparatus 110 from the storage circuitry 112. In this case, the image 31 captured in preparatory imaging is, for example, an image having a lower resolution than that of an image captured in main imaging. The image 31 captured in preparatory imaging is, for example, an image having a wider imaging range than that of an image captured in main imaging.

The acquisition function 116a acquires finding information obtained through a medical interview, a physical examination, and other tests of the subject from the RIS 130, the PACS 120, the interpretation report system 150, and the like.

The setting function 116b sets the main imaging region including a plurality of slices to be captured in main imaging based on the image that is captured in preparatory imaging and acquired by the acquisition function 116a.

For example, as illustrated in FIG. 3B, the setting function 116b displays the image 31 captured in preparatory imaging of the subject on the display 114, and receives an operation of designating an imaging range 32 of main imaging on the displayed image 31 captured in preparatory imaging through the input interface 113. Further, the setting function 116b sets the imaging range 32 designated by the operator as the main imaging region.

Alternatively, the setting function 116b may automatically set the main imaging region from the image 31 captured in preparatory imaging. For example, the setting function 116b may set the main imaging region by inputting the image 31 captured in preparatory imaging of the subject to be diagnosed to a machine learning model trained, by inputting an image captured in preparatory imaging of any subject, to estimate and output the main imaging region based on the image captured in preparatory imaging using a machine learning method such as deep learning. Further, for example, the setting function 116b may extract a characteristic site from the image captured in preparatory imaging of the subject using a known image processing method such as segmentation processing, and may set the main imaging region based on the extracted site.

The identification function 116c identifies the position of an abnormal slice from among the plurality of slices included in the main imaging region set by the setting function 116b based on the image captured in preparatory imaging and finding information acquired by the acquisition function 116a.

For example, as illustrated in FIG. 3B, the identification function 116c displays finding information obtained through a medical interview, a physical examination, and other tests of the subject on the display 114 (not illustrated), and receives, from the operator, an operation of placing a line 33 indicating the position of the abnormal slice at a position of a site with a suspicion of disease indicated by finding information on the image 31 that is captured in preparatory imaging and is displayed on the display 114 via the input interface 113. Further, the identification function 116c identifies the position of the line 33 placed by the operator as the position of the abnormal slice.

Alternatively, the identification function 116c may automatically identify the position of the abnormal slice based on the image 31 captured in preparatory imaging and finding information. For example, the identification function 116c may identify the position of the abnormal slice by inputting the image 31 captured in preparatory imaging of the subject to be diagnosed and finding information to a machine learning model trained, by inputting an image captured in preparatory imaging of any subject and finding information, to output the position of the abnormal slice based on the image captured in preparatory imaging and the finding information using a machine learning method such as deep learning. Further, for example, the setting function 116b may extract a site with a suspicion of disease estimated based on a medical interview and a physical examination included in finding information from the image captured in preparatory imaging of the subject using a known image processing method such as segmentation processing, and may identify the position of the abnormal slice based on the extracted site.

The imaging control function 116d performs main imaging to capture an image of the main imaging region set by the setting function 116b.

Specifically, the imaging control function 116d controls the imaging unit 115 to capture an image of the main imaging region based on imaging conditions input from the operator, thereby generating the image captured in main imaging.

The output function 116e outputs the image captured in main imaging together with abnormal slice information about the position of the abnormal slice identified by the identification function 116c.

For example, as illustrated in FIG. 3B, the output function 116e outputs the image captured in main imaging by adding, as the abnormal slice information, an image with a slice line corresponding to the position of the abnormal slice on the image captured in preparatory imaging, a slice number (numerical value) for identifying the abnormal slice from among the plurality of slices, a direction (left-right, up-down, front-back) of the abnormal slice, anatomical information about an abnormal site included in the abnormal slice, and the like to the image captured in main imaging.

The display control function 116f identifies an image of the abnormal slice from among images of the plurality of slices included in the image captured in main imaging based on the abnormal slice information output from the output function 116e, and initially displays the identified image of the abnormal slice on the display 114.

For example, as illustrated in FIG. 3C, the display control function 116f identifies an image of a slice at the position of the line 33 placed on the image 31 captured in preparatory imaging by the operator from among the images of the plurality of slices included in the image captured in main imaging based on the abnormal slice information. The display control function 116f initially displays the identified image as an abnormal slice image 34 on the display 114.

According to the configuration described above, the position of the abnormal slice is identified based on the image captured in preparatory imaging and finding information, and the image captured in main imaging is output together with the identified position of the abnormal slice, thereby making it possible to reduce the time required for identifying the abnormal slice.

While the processing functions of the processing circuitry 116 of the medical image diagnostic apparatus 110 have been described above, the medical image diagnostic apparatus 110 according to the present embodiment can be applied to various imaging examinations depending on a disease and the like. Specific applications of the medical image diagnostic apparatus 110 according to the present embodiment will be described below as examples.

### First Example

A first example is an example where the medical image processing system 100 is an MRI apparatus, a positioning image is used as the image captured in preparatory imaging, and a physical finding is used as the finding information.

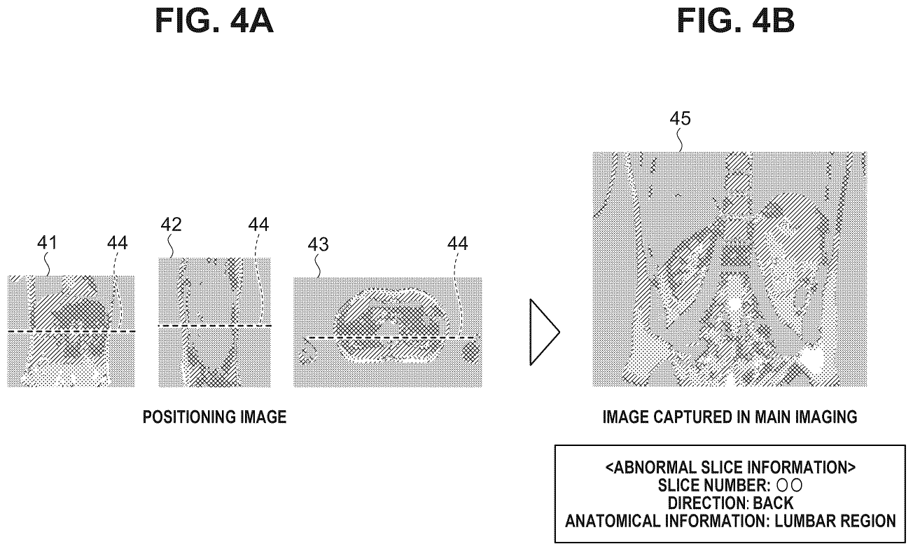

FIGS. 4A and 4B each illustrate the first example of the medical image diagnostic apparatus 110 according to the first embodiment.

In the present example, the acquisition function 116a acquires a positioning image captured before main imaging in a same imaging examination of a subject as an image captured in preparatory imaging. For example, the acquisition function 116a acquires the positioning image captured in preparatory imaging of the subject by the medical image diagnostic apparatus 110 from the storage circuitry 112. The acquisition function 116a may also acquire, as the image captured in preparatory imaging, a magnetic-field adjustment image, a sensitivity map image, or the like captured before the main imaging in the same imaging examination of the subject.

For example, as illustrated in FIG. 4A, the acquisition function 116a acquires a coronal section image 41, a sagittal section image 42, or an axial cross-sectional image 43 captured as the positioning image in preparatory imaging of the subject.

Further, the acquisition function 116a acquires, as the finding information, a physical finding obtained through a medical interview and a physical examination of the subject. For example, the acquisition function 116a acquires the physical finding about the subject from the RIS 130 and the like. For example, the physical finding refers to information about pain, swelling, or the like acquired through visual inspection, palpation, auscultation, percussion, or the like performed on the subject. Alternatively, the physical finding may be information about the body obtained from the subject during an examination setup by an operator of an imaging examination.

Further, the setting function 116b sets the main imaging region including a plurality of slices to be captured in main imaging based on the positioning image acquired by the acquisition function 116a.

The identification function 116c identifies the position of an abnormal slice from among the plurality of slices included in the main imaging region set by the setting function 116b based on the positioning image and the physical finding acquired by the acquisition function 116a.

For example, as illustrated in FIG. 4A, when the contents of the physical finding indicate that the subject has complained of pain in a lumbar region during a medical interview, the identification function 116c receives, from the operator, an operation of placing a line 44 at the position of the lumbar region on the coronal section image 41, the sagittal section image 42, or the axial cross-sectional image 43. Further, the identification function 116c identifies the position of the line 44 placed by the operator as the position of the abnormal slice.

The imaging control function 116d performs main imaging to capture an image of the main imaging region set by the setting function 116b.

The output function 116e outputs the image captured in main imaging together with the abnormal slice information identified by the identification function 116c.

For example, as illustrated in FIG. 4B, the output function 116e outputs an image 45 captured in main imaging by adding, as the abnormal slice information, the slice number (e.g., ○○) of the abnormal slice, the direction (e.g., back) of the abnormal slice, the anatomical information (e.g., lumbar region) about an abnormal site included in the abnormal slice, and the like to the image 45 captured in main imaging.

### Second Example

A second example is an example where the medical image processing system 100 is an X-ray CT apparatus, a positioning image is used as the image captured in preparatory imaging, and a physical finding is used as the finding information.

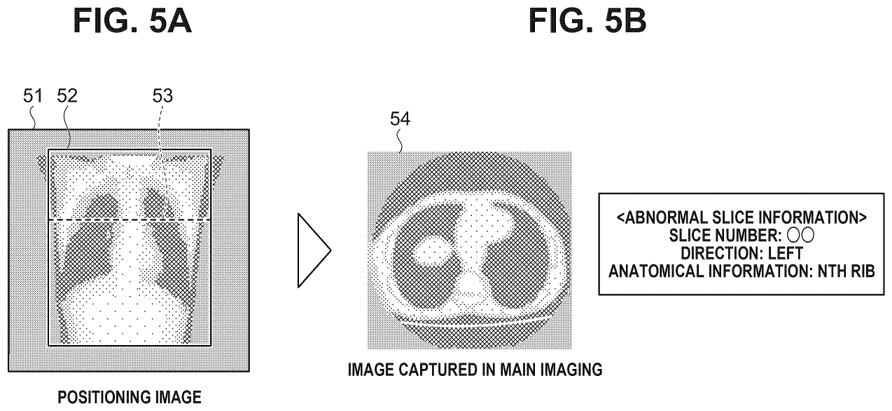

FIGS. 5A and 5B each illustrate the second example of the medical image diagnostic apparatus 110 according to the first embodiment.

In the present example, the acquisition function 116a acquires a positioning image captured before main imaging in a same imaging examination of a subject as an image captured in preparatory imaging. For example, the acquisition function 116a acquires the positioning image captured in preparatory imaging of the subject by the medical image diagnostic apparatus 110 from the storage circuitry 112.

For example, as illustrated in FIG. 5A, the acquisition function 116a acquires a scanogram image 51 captured as the positioning image in preparatory imaging of the subject.

Further, the acquisition function 116a acquires, as the finding information, a physical finding obtained through a medical interview and a physical examination of the subject. For example, the acquisition function 116a acquires the physical finding about the subject from the RIS 130 and the like.

Further, the setting function 116b sets the main imaging region including a plurality of slices to be captured in main imaging based on the positioning image acquired by the acquisition function 116a.

For example, as illustrated in FIG. 5A, the setting function 116b receives, from the operator, an operation of designating an imaging range 52 of main imaging on the scanogram image 51. Further, the setting function 116b sets the imaging range 52 designated by the operator as the main imaging region.

The identification function 116c identifies the position of an abnormal slice from among the plurality of slices included in the main imaging region set by the setting function 116b based on the positioning image and the physical finding acquired by the acquisition function 116a.

For example, as illustrated in FIG. 5A, when the contents of the physical finding indicate a suspected fracture in the left rib, the identification function 116c receives, from the operator, an operation of placing a line 53 at the position of the suspected fracture in the left rib on the scanogram image 51. Further, the identification function 116c identifies the position of the line 53 placed by the operator as the position of the abnormal slice.

The imaging control function 116d performs main imaging to capture an image of the main imaging region set by the setting function 116b.

The output function 116e outputs the image captured in main imaging together with the abnormal slice information identified by the identification function 116c

For example, as illustrated in FIG. 5B, the output function 116e outputs an image 54 captured in main imaging by adding, as the abnormal slice information, the slice number (e.g., ○○) of the abnormal slice, the direction (e.g., left) of the abnormal slice, the anatomical information (e.g., Nth rib) about an abnormal site included in the abnormal slice, and the like to the image 54 captured in main imaging.

### Third Example

A third example is an example where the medical image processing system 100 is an X-ray CT apparatus, a positioning image is used as the image captured in preparatory imaging, and a test finding obtained through another imaging examination is used as the finding information.

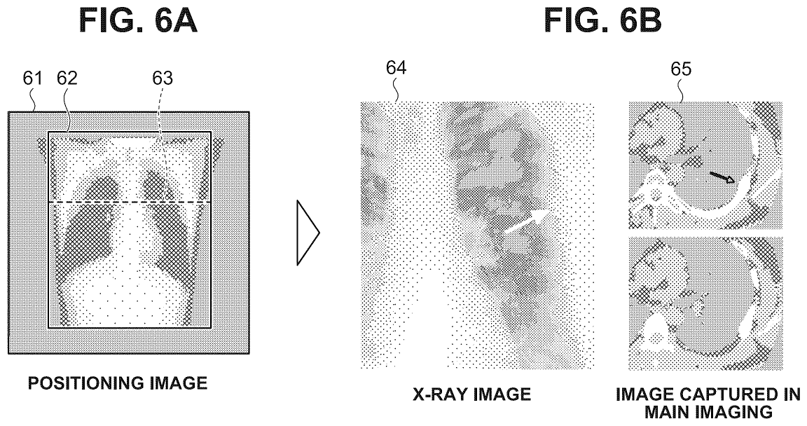

FIGS. 6A and 6B each illustrate the third example of the medical image diagnostic apparatus 110 according to the first embodiment.

In the present example, acquisition function 116a acquires a positioning image captured before main imaging in a same imaging examination of a subject as an image captured in preparatory imaging. For example, the acquisition function 116a acquires the positioning image captured in preparatory imaging of the subject by the medical image diagnostic apparatus 110 from the storage circuitry 112.

For example, as illustrated in FIG. 6A, the acquisition function 116a acquires a scanogram image 61 captured as the positioning image in preparatory imaging of the subject.

Further, the acquisition function 116a acquires a medical image captured by another medical image diagnostic apparatus in another imaging examination of the subject. For example, the acquisition function 116a acquires a medical image of a cross-section similar to the positioning image captured in the current imaging examination. In this case, the medical image may be an image captured in main imaging in the other imaging examination, or may be an image captured in preparatory imaging.

For example, as illustrated in FIG. 6B, the acquisition function 116a acquires an X-ray image captured by an X-ray diagnostic apparatus in another imaging examination of the subject. The acquisition function 116a may acquire a CT image captured by an X-ray CT apparatus, an MR image captured by an MRI apparatus, a PET image captured by a PET apparatus, a SPECT image captured by a SPECT apparatus, an ultrasound image captured by an ultrasonic diagnostic apparatus, and the like in the other imaging examination of the subject.

Further, the acquisition function 116a acquires, as the finding information, a test finding obtained through the other imaging examination of the subject. For example, the acquisition function 116a acquires the test finding from supplementary information about the medical image captured in the other imaging examination of the subject. Alternatively, for example, the acquisition function 116a may acquire a test finding obtained through the other imaging examination of the subject from the interpretation report system 150 and the like.

Further, the setting function 116b sets the main imaging region including a plurality of slices to be captured in main imaging based on the positioning image acquired by the acquisition function 116a.

For example, as illustrated in FIG. 6A, the setting function 116b receives, from the operator, an operation of designating an imaging range 62 of main imaging on the scanogram image 61. Further, the setting function 116b sets the imaging range 62 designated by the operator as the main imaging region.

The identification function 116c identifies the position of an abnormal slice from among the plurality of slices included in the main imaging region set by the setting function 116b based on the positioning image acquired by the acquisition function 116a, a test finding obtained through the other imaging examination, and the medical image captured by the other medical image diagnostic apparatus in the other imaging examination.

For example, as illustrated in FIGS. 6A and 6B, the identification function 116c displays the scanogram image 61 captured as the positioning image, a test finding (not illustrated) obtained through the other imaging examination, and an X-ray image 64 captured by the other medical image diagnostic apparatus on the display 114, and receives, from the operator, an operation of placing a line 63 at the position of a site with a suspicion of disease indicated by the test finding and the X-ray image 64 on the scanogram image 61. Further, the identification function 116c identifies the position of the line 63 placed by the operator as the position of the abnormal slice.

The imaging control function 116d performs main imaging to capture an image of the main imaging region set by the setting function 116b.

The output function 116e outputs the image captured in main imaging together with the abnormal slice information identified by the identification function 116c.

For example, as illustrated in FIG. 6B, the output function 116e outputs an image 65 that is a CT image captured in main imaging together with abnormal slice information (not illustrated).

### Fourth Example

A fourth example is an example where a positioning image is used as the image captured in preparatory imaging, and a physical finding and a test finding obtained through a blood test are used as the finding information.

In the present example, the acquisition function 116a acquires a positioning image captured before main imaging in a same imaging examination of a subject as an image captured in preparatory imaging. For example, the acquisition function 116a acquires the positioning image captured in preparatory imaging of the subject by the medical image diagnostic apparatus 110 from the storage circuitry 112.

Further, the acquisition function 116a acquires, as the finding information, a physical finding obtained through a medical interview and a physical examination of the subject. For example, the acquisition function 116a acquires the physical finding about the subject from the RIS 130 and the like.

Further, the acquisition function 116a acquires, as the finding information, a test finding obtained through a blood test of the subject. For example, the acquisition function 116a acquires a test finding obtained through the blood test of the subject from the interpretation report system 150 and the like. The acquisition function 116a may also acquire a past medical history, a family medical history, or the like of the subject as the finding information.

Further, the setting function 116b sets the main imaging region including a plurality of slices to be captured in main imaging based on the positioning image acquired by the acquisition function 116a.

The identification function 116c identifies the position of an abnormal slice from among the plurality of slices included in the main imaging region set by the setting function 116b based on the positioning image and the physical finding acquired by the acquisition function 116a, and a test finding obtained through a blood test.

For example, when contents of the physical finding indicate that the subject has complained of pain in an upper left part of the back during a medical interview, and the test finding obtained through a blood test indicates that the subject is suspected of hypersplenism, the identification function 116c receives an operation of placing a line at the position of the spleen on the positioning image. Further, the identification function 116c identifies the position of the line placed by the operator as the position of the abnormal slice.

The imaging control function 116d performs main imaging to capture an image of the main imaging region set by the setting function 116b.

The output function 116e outputs the image captured in main imaging together with the abnormal slice information identified by the identification function 116c.

For example, the output function 116e outputs the image captured in main imaging by adding, as the abnormal slice information, the slice number (e.g., ○○) of the abnormal slice, the direction (e.g., back) of the abnormal slice, the anatomical information (e.g., spleen) about an abnormal site included in the abnormal slice, and the like to the image captured in main imaging.

### Fifth Example

A fifth example is an example where a positioning image is used as the image captured in preparatory imaging and an optical camera image is used as the finding information.

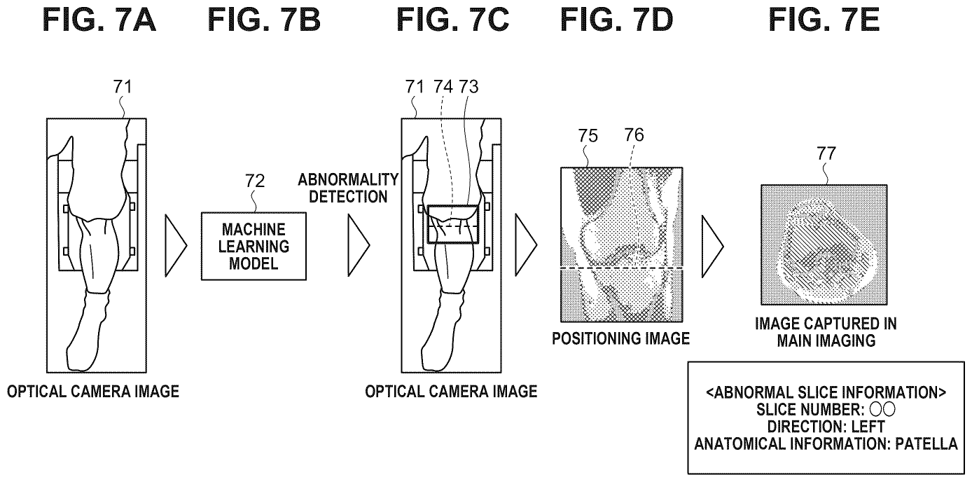

FIGS. 7A to 7E each illustrate the fifth example of the medical image diagnostic apparatus 110 according to the first embodiment.

In the present example, the acquisition function 116a acquires a positioning image captured before main imaging in a same imaging examination of a subject as an image captured in preparatory imaging. For example, the acquisition function 116a acquires the positioning image captured in preparatory imaging of the subject by the medical image diagnostic apparatus 110 from the storage circuitry 112.

Further, the acquisition function 116a acquires an optical camera image of the subject as the finding information. For example, the acquisition function 116a acquires an optical camera image of the subject that is captured by a camera provided in the medical image diagnostic apparatus 110, an imaging room, or the like.

For example, as illustrated in FIG. 7A, the acquisition function 116a acquires an optical camera image 71 of a left leg portion of the subject.

Further, the setting function 116b sets the main imaging region including a plurality of slices to be captured in main imaging based on the positioning image acquired by the acquisition function 116a.

The identification function 116c detects an abnormal portion included in the optical camera image of the subject acquired by the acquisition function 116a using a machine learning model trained to, by inputting an optical camera image of any subject, detect and output an abnormal portion included in the optical camera image, and identifies the position of the abnormal slice based on the detected abnormal portion and the positioning image.

For example, as illustrated in FIGS. 7B and 7C, the identification function 116c detects an abnormal site 73 and an abnormal position 74 included in the optical camera image 71 by inputting the optical camera image 71 to a trained machine learning model 72 trained to, by inputting an optical camera image of any subject, output an abnormal site and an abnormal position included in the optical camera image. For example, in a case where the optical camera image 71 is an image of a left leg portion of the subject, the patella of the left leg portion is detected as the abnormal site 73, and the position where an abnormality has occurred in the patella of the left leg portion is detected as the abnormal position 74.

For example, as illustrated in FIG. 7D, the identification function 116c receives, from the operator, an operation of placing a line 76 at the position indicated by the abnormal position 74 detected from the optical camera image 71 on a positioning image 75. Further, the identification function 116c identifies the position of the line 76 placed by the operator as the position of the abnormal slice.

The imaging control function 116d performs main imaging to capture an image of the main imaging region set by the setting function 116b.

The output function 116e outputs the image captured in main imaging together with the abnormal slice information identified by the identification function 116c.

For example, as illustrated in FIG. 7E, the output function 116e outputs an image 77 captured in main imaging by adding, as the abnormal slice information, the slice number (e.g., ○○) of the abnormal slice, the direction (e.g., left) of the abnormal slice, the anatomical information (e.g., patella) about an abnormal site included in the abnormal slice, and the like to the image 77 captured in main imaging.

In this example, however, as in the examples described above, a physical finding obtained through a medical interview and a physical examination of the subject may also be used as the finding information.

### Sixth Example

A sixth example is an example where a virtual positioning image estimated based on an optical camera image is used as the image captured in preparatory imaging, and a physical findings are used as the finding information.

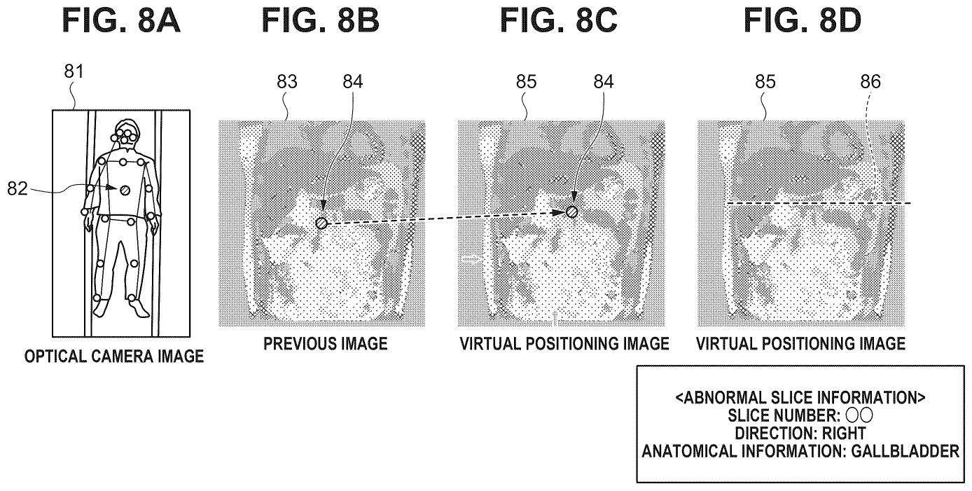

FIGS. 8A to 8D each illustrate the sixth example of the medical image diagnostic apparatus 110 according to the first embodiment.

In the sixth example, the acquisition function 116a acquires, as the image captured in preparatory imaging, a virtual positioning image estimated based on an optical camera image of a subject.

Specifically, the acquisition function 116a acquires an optical camera image of the subject. For example, the acquisition function 116a acquires the optical camera image 71 of the subject that is captured by a camera provided in the medical image diagnostic apparatus 110, an imaging room, or the like. For example, the acquisition function 116a acquires the optical camera image of the subject that is captured by the camera provided in the medical image diagnostic apparatus 110, the imaging room, or the like. Then, the acquisition function 116a estimates the position of a predetermined characteristic site from the acquired optical camera image.

For example, as illustrated in FIG. 8A, the acquisition function 116a acquires an optical camera image 81 of the whole body of the subject, and estimates a position 82 of an abdominal region from the acquired optical camera image 81.

Further, the acquisition function 116a acquires the positioning image of the subject that is previously captured. For example, the acquisition function 116a acquires the previous positioning image of the subject from the PACS 120 and the like. Further, the acquisition function 116a estimates the position of a characteristic site that is identical to the characteristic site estimated based on the optical camera image from the acquired previous positioning image of the subject.

For example, as illustrated in FIG. 8B, the acquisition function 116a acquires a previously captured positioning image 83 of the subject and estimates a position 84 of the abdominal region from the acquired positioning image 83.

Further, the acquisition function 116a acquires a virtual positioning image by moving the previous positioning image so that the estimated positions of the characteristic sites match based on a positional change between the position of the characteristic site estimated from the optical camera image and the position of the characteristic site estimated from the previous positioning image. Alternatively, the acquisition function 116a may acquire the virtual positioning image from the optical camera image of the subject and the previous positioning image by using linear transformation or a machine learning model.

For example, as illustrated in FIG. 8C, the acquisition function 116a acquires a virtual positioning image 85 by translating the previous positioning image 83 in a head-to-tail direction and a left-right direction so as to align the position of the abdominal region based on a positional change between the position 82 of the abdominal region estimated from the optical camera image 81 and the position 84 of the abdominal region estimated from the previous positioning image 83.

Further, the acquisition function 116a acquires, as the finding information, a physical finding obtained through a medical interview and a physical examination of the subject. For example, the acquisition function 116a acquires the physical finding about the subject from the RIS 130 and the like.

Further, the setting function 116b sets the main imaging region including a plurality of slices to be captured in main imaging based on the virtual positioning image acquired by the acquisition function 116a.

The identification function 116c identifies the position of an abnormal slice from among the plurality of slices included in the main imaging region set by the setting function 116b based on the virtual positioning image and the physical finding acquired by the acquisition function 116a.

For example, as illustrated in FIG. 8D, when the contents of the physical finding indicate a suspicion of cholecystitis, the identification function 116c receives, from the operator, an operation of placing a line 86 at the position of the gallbladder on the virtual positioning image 85. Further, the identification function 116c identifies the position of the line 86 placed by the operator as the position of the abnormal slice.

The imaging control function 116d performs main imaging to capture an image of the main imaging region set by the setting function 116b.

The output function 116e outputs the image captured in main imaging together with abnormal slice information identified by the identification function 116c.

For example, as illustrated in FIG. 8D, the output function 116e outputs the image captured in main imaging (not illustrated) by adding, as the abnormal slice information, the slice number (e.g., ○○) of the abnormal slice, the direction (e.g., right) of the abnormal slice, the anatomical information (e.g., gallbladder) about an abnormal site included in the abnormal slice, and the like to the image captured in main imaging.

In this example, since a virtual positioning image estimated based on an optical camera image of the subject is used as the image captured in preparatory imaging, capturing a positioning image in preparatory imaging is not necessary.

### Seventh Example

A seventh example is an example where the medical image processing system 100 is an MRI apparatus, a positioning image is used as the image captured in preparatory imaging, a physical finding is used as the finding information, and an MR spectroscopy image of multiple voxels is captured in main imaging.

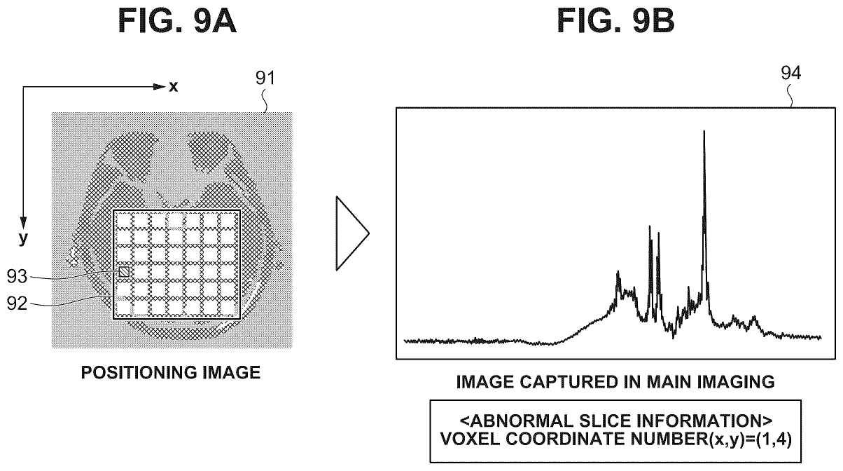

FIGS. 9A and 9B each illustrate the seventh example of the medical image diagnostic apparatus 110 according to the first embodiment.

In the present example, the acquisition function 116a acquires a positioning image captured before main imaging in a same imaging examination of a subject as an image captured in preparatory imaging. For example, the acquisition function 116a acquires the positioning image captured in preparatory imaging of the subject by the medical image diagnostic apparatus 110 from the storage circuitry 112.

Further, the acquisition function 116a acquires, as the finding information, a physical finding obtained through a medical interview and a physical examination of the subject. For example, the acquisition function 116a acquires the physical finding about the subject from the RIS 130 and the like.

Further, the setting function 116b sets the main imaging region including a plurality of slices to be captured in main imaging based on the positioning image acquired by the acquisition function 116a.

For example, as illustrated in FIG. 9A, the setting function 116b receives, from the operator, an operation of designating an imaging range 92 of main imaging on a positioning image 91. Further, the setting function 116b sets the imaging range 92 designated by the operator as the main imaging region.

The identification function 116c identifies the position of an abnormal slice from among the plurality of slices included in the main imaging region set by the setting function 116b based on the positioning image and the physical finding acquired by the acquisition function 116a.

For example, as illustrated in FIG. 9A, the identification function 116c displays the physical finding obtained through a medical interview and a physical examination of the subject on the display 114 (not illustrated), and receives, from the operator, an operation of designating a voxel 93 located at a position of a site with a suspicion of disease indicated by the physical finding on the positioning image 91. Further, the identification function 116c identifies the position of the voxel 93 designated by the operator as the position of the abnormal slice.

The imaging control function 116d performs main imaging to capture an MR spectroscopy image of each voxel included in the main imaging region set by the setting function 116b.

The output function 116e outputs the MR spectroscopy image captured in main imaging together with abnormal slice information identified by the identification function 116c.

For example, as illustrated in FIG. 9B, the output function 116e outputs an MR spectroscopy image 94 captured in main imaging by adding, as the abnormal slice information, a voxel coordinate number (e.g., (x, y)=(1, 4)) corresponding to an abnormal portion, and the like to the MR spectroscopy image 94 captured in main imaging.

While the processing functions included in the processing circuitry 116 of the medical image diagnostic apparatus 110 have been described above, in the present embodiment, the processing circuitry 116 is implemented by, for example, a processor. In this case, the processing functions included in the processing circuitry 116 are stored in the storage circuitry 112 in the form of, for example, programs that can be executed by a computer. The processing circuitry 116 reads out each program from the storage circuitry 112 and executes the program, thereby implementing the processing function corresponding to the program. In other words, the processing circuitry 116 has the processing functions illustrated in FIG. 2 in a state where the programs are read out.

A procedure of processing to be performed by each processing function included in the processing circuitry 116 of the medical image diagnostic apparatus 110 will be described below.

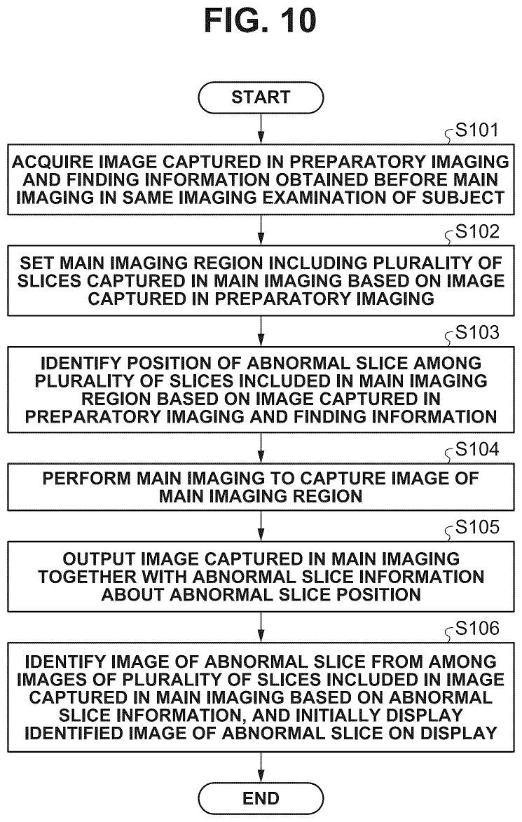

FIG. 10 is a flowchart illustrating a procedure of processing to be performed by each processing function included in the processing circuitry 116 of the medical image diagnostic apparatus 110 according to the first embodiment.

For example, as illustrated in FIG. 10, in the first embodiment, in step S101, the acquisition function 116a acquires an image captured in preparatory imaging and finding information obtained before main imaging in a same imaging examination of a subject. The processing in step S101 is implemented, for example, by the processing circuitry 116 reading out a program corresponding to the acquisition function 116a from the storage circuitry 112 and executing the program.

Next, in step S102, the setting function 116b sets the main imaging region including a plurality of slices to be captured in main imaging based on the image that is captured in preparatory imaging and is acquired by the acquisition function 116a. The processing in step S102 is implemented, for example, by the processing circuitry 116 reading out a program corresponding to the setting function 116b from the storage circuitry 112 and executing the program.

Next, in step S103, the identification function 116c identifies the position of an abnormal slice from among the plurality of slices included in the main imaging region set by the setting function 116b based on the image captured in preparatory imaging and the finding information acquired by the acquisition function 116a. The processing in step S103 is implemented, for example, by the processing circuitry 116 reading out a program corresponding to the identification function 116c from the storage circuitry 112 and executing the program.

Next, in step S104, the imaging control function 116d performs main imaging to capture an image of the main imaging region set by the setting function 116b. The processing in step S104 is implemented, for example, by the processing circuitry 116 reading out a program corresponding to the imaging control function 116d from the storage circuitry 112 and executing the program.

Next, in step S105, the output function 116e outputs the image captured in main imaging together with abnormal slice information about the position of the abnormal slice identified by the identification function 116c. The processing in step S105 is implemented, for example, by the processing circuitry 116 reading out a program corresponding to the output function 116e from the storage circuitry 112 and executing the program.

Next, in step S106, the display control function 116f identifies an image of the abnormal slice from among images of the plurality of slices included in the image captured in main imaging based on the abnormal slice information output from the output function 116e, and initially displays the identified image of the abnormal slice on the display 114. The processing in step S106 is implemented, for example, by the processing circuitry 116 reading out a program corresponding to the display control function 116f from the storage circuitry 112 and executing the program.

As described above, in the first embodiment, the acquisition function 116a acquires the image captured in preparatory imaging and finding information obtained before main imaging in a same imaging examination of the subject. The setting function 116b sets the main imaging region including a plurality of slices to be captured in main imaging based on the image captured in preparatory imaging. The identification function 116c identifies the position of an abnormal slice from among the plurality of slices included in the main imaging region based on the image captured in preparatory imaging and the finding information. The imaging control function 116d performs main imaging to capture an image of the main imaging region. The output function 116e outputs the image captured in main imaging together with abnormal slice information indicating the position of the abnormal slice.

Thus, according to the first embodiment, the position of an abnormal slice is identified based on an image captured in preparatory imaging and finding information, and the image captured in main imaging is output together with the identified position of the abnormal slice, thereby it is possible to reduce the time required for identifying the abnormal slice.

In the first embodiment, the display control function 116f identifies the image of the abnormal slice from among the images of the plurality of slices included in the image captured in main imaging based on the abnormal slice information, and initially displays the identified image of the abnormal slice on the display 114.

Thus, according to the first embodiment, the image of the abnormal slice is automatically displayed, which saves the time and labor to search for a slice including an abnormal site in the image captured in main imaging, and it is possible to further reduce the time required for identifying the abnormal slice.

While the first embodiment has been described above, the medical image diagnostic apparatus 110 described above can also be implemented by changing a part of the configuration thereof as appropriate. Thus, a modified example of the medical image diagnostic apparatus 110 according to the first embodiment will be described below as another embodiment. In the following embodiment, differences from the above-described embodiment will be mainly described, and constituent elements having the same functions are denoted by the same reference numerals, and detailed descriptions thereof will be omitted.

### Second Embodiment

For example, in the above-described embodiment, in the medical image diagnostic apparatus 110, the display control function 116f identifies an image of an abnormal slice from among images of the plurality of slices included in the image captured in main imaging based on abnormal slice information output from the output function 116e, and initially displays the identified image of the abnormal slice on the display 114. However, the embodiment is not limited thereto.

For example, the output function 116e may transmit the abnormal slice information to the interpretation terminal device 140.

In this case, the interpretation terminal device 140 receives the abnormal slice information output from the output function 116e via the network 160, and further acquires the image captured in main imaging from the medical image diagnostic apparatus 110. Further, the interpretation terminal device 140 identifies an image of an abnormal slice from among images of the plurality of slices included in the image captured in main imaging based on the received abnormal slice information, and initially displays the identified image of the abnormal slice on a display.

For example, the output function 116e may transmit the abnormal slice information to the RIS 130 or the interpretation report system 150 as information to be entered in a comment field of report information.

In this case, the RIS 130 or the interpretation report system 150 receives the abnormal slice information output from the output function 116e via the network 160, and further acquires the image captured in main imaging from the medical image diagnostic apparatus 110. Further, the interpretation terminal device 140 updates the comment field of the report information about the image captured in main imaging based on the received abnormal slice information.

### Other Embodiments

While the above-described embodiments illustrate an example where the acquisition unit, the setting unit, the identification unit, the imaging control unit, the output unit, and the display control unit according to the present specification are respectively implemented by the setting function, the identification function, the imaging control function, the output function, and the display control function of processing circuitry, the embodiments are not limited thereto. For example, instead of implementing the setting unit, the identification unit, the imaging control unit, the output unit, and the display control unit according to the present specification by the setting function, the identification function, the imaging control function, the output function, and the display control function, respectively, which are described above in the embodiments, the setting unit, the identification unit, the imaging control unit, the output unit, and the display control unit may be implemented by hardware only, software only, or a combination of hardware and software.

While, in the above-described embodiments, the processing circuitry is implemented by a single processor, the embodiments are not limited thereto. For example, the processing circuitry may include a combination of a plurality of independent processors, and each processing function may be implemented by each processor executing a program. Further, processing functions of the processing circuitry may be implemented by being distributed among a plurality of pieces of processing circuitry or integrated into a single piece of processing circuitry as appropriate. Further, the processing functions of the processing circuitry may be implemented by a combination of hardware such as circuitry and software. In this case, programs corresponding to the respective processing functions are stored in a single storage circuitry. However, the embodiments are not limited thereto. For example, the programs corresponding to the respective processing functions may be stored in such a manner that the programs are distributed into a plurality of pieces of storage circuitry and the processing circuitry may be configured to read out each program from the corresponding storage circuitry and execute the program.

The term “processor” used in the embodiments described above refers to, for example, circuitry of a central processing unit (CPU), a graphics processing unit (GPU), an application specific integrated circuit (ASIC), a programmable logic device (e.g., a simple programmable logic device (SPLD), a complex programmable logic device (CPLD), and a field programmable gate array (FPGA)), or the like. Instead of storing each program in the storage circuitry, each program may be directly incorporated into circuitry of a processor. In this case, the processor implements each function by reading out each program incorporated in the circuitry and executing the program. The configuration of each processor according to the present embodiment is not limited to a configuration as a single piece of circuitry for each processor, but instead may be a configuration as a plurality of pieces of independent circuitry combined to form one processor to thereby implement a function.

Programs to be executed by the processor are provided by being incorporated in advance in a read-only memory (ROM), storage circuitry, or the like. The programs may be provided by being recorded on a computer-readable storage medium such as a compact-disk (CD)-ROM, a flexible disk (FD), a CD-recordable (R), or a digital versatile disk (DVD) in a file in a format that is installable to or executable by these devices. Further, the programs may be stored in a computer connected to a network such as the Internet so as to be provided or distributed by being downloaded via the network. For example, the programs are configured with modules including the functional units described above. In the actual hardware, by a CPU reading and executing the programs from a storage medium such as a ROM, the modules are loaded into a main storage device and generated in the main storage device.

The constituent elements of the apparatuses and devices illustrated in the above-described embodiments are functional and conceptual in nature. Thus, it is not necessary that the constituent elements be physically configured as illustrated in the drawings. In other words, specific modes of distribution and integration of the apparatuses and devices are not limited to those illustrated in the drawings. It is possible to functionally or physically distribute or integrate all or a part of the apparatuses and devices in any units depending on various loads, the status of use, and the like. Further, all or any part of the processing functions performed by the apparatuses and devices may be implemented by a CPU and a program analyzed and executed by the CPU or may be implemented as hardware by wired logic.

With regard to the processes described in the above-described embodiments, it is also possible to manually perform all or a part of the processes described as being performed automatically. Alternatively, by using a known method, it is also possible to automatically perform all or a part of the processes described as being performed manually. In addition, unless otherwise noted, it is possible to freely modify a processing procedure, a controlling procedure, a specific name, and information including various kinds of data and parameters that are described above and illustrated in the drawings.

According to at least one of the embodiments described above, it is possible to reduce the time required for identifying an abnormal slice.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image diagnostic apparatus comprising processing circuitry configured to:
acquire an image captured in preparatory imaging and finding information obtained before main imaging in an imaging examination of a subject;
set a main imaging region including a plurality of slices to be captured in the main imaging based on the image captured in the preparatory imaging;
identify a position of an abnormal slice from among the plurality of slices included in the main imaging region based on the image captured in the preparatory imaging and the finding information;
perform the main imaging to capture an image of the main imaging region; and
output the image captured in the main imaging together with abnormal slice information about the position of the abnormal slice.

2. The medical image diagnostic apparatus according to claim 1, wherein the medical image diagnostic apparatus is one of a magnetic resonance imaging (MRI) apparatus, an X-ray computed tomography (CT) apparatus, a positron emission tomography (PET)-CT apparatus, and a single photon emission computed tomography (SPECT)-CT apparatus.

3. The medical image diagnostic apparatus according to claim 1, wherein the processing circuitry is further configured to output the image captured in the main imaging by adding, as the abnormal slice information, at least one of an image with a slice line corresponding to the position of the abnormal slice on the image captured in the preparatory imaging, a slice number for identifying the abnormal slice from among the plurality of slices, a direction of the abnormal slice, anatomical information about an abnormal site included in the abnormal slice, and a coordinate number for a voxel at an abnormal portion to the image captured in the main imaging.

4. The medical image diagnostic apparatus according to claim 1, wherein the processing circuitry is further configured to identify an image of the abnormal slice from among images of the plurality of slices included in the image captured in the main imaging based on the abnormal slice information, and to initially display the identified image of the abnormal slice on a display.

5. The medical image diagnostic apparatus according to claim 1, wherein the processing circuitry is further configured to transmit the abnormal slice information to an interpretation terminal device.

6. The medical image diagnostic apparatus according to claim 1, wherein the processing circuitry is further configured to transmit the abnormal slice information to either a radiology information system or an interpretation report system as information to be entered in a comment field of report information.

7. The medical image diagnostic apparatus according to claim 1, wherein the processing circuitry is further configured to acquire, as the image captured in the preparatory imaging, a virtual positioning image estimated based on a positioning image captured before the main imaging, a magnetic-field adjustment image or a sensitivity map image to be used for a magnetic resonance imaging apparatus, or an optical camera image.

8. The medical image diagnostic apparatus according to claim 1, wherein the processing circuitry is further configured to acquire, as the finding information, a physical finding, a test finding, a past medical history, or a family medical history of the subject.

9. The medical image diagnostic apparatus according to claim 1, wherein the processing circuitry is further configured to:
acquire a medical image captured by another medical image diagnostic apparatus in another imaging examination of the subject; and
identify the position of the abnormal slice further based on the medical image captured by the other medical image diagnostic apparatus.

10. The medical image diagnostic apparatus according to claim 1, wherein the image captured in the preparatory imaging has a lower resolution than the image captured in the main imaging.

11. The medical image diagnostic apparatus according to claim 1, wherein an imaging range of the image captured in the preparatory imaging is wider than an imaging range of the image captured in the main imaging.

12. The medical image diagnostic apparatus according to claim 1, wherein the processing circuitry is further configured to:
acquire, as the finding information, an optical camera image of the subject; and
detect an abnormal portion included in the optical camera image of the subject using a machine learning model trained, by inputting an optical camera image of any subject, to output an abnormal portion included in the optical camera image, and identify the position of the abnormal slice based on the detected abnormal portion and the image captured in the preparatory imaging.
